(19) United States
(12) Patent Application Publication
(10) Pub. No.: US 2026/0151449 A1
(43) Pub. Date: Jun. 4, 2026

(54) TOPICAL COMPOSITION COMPRISING AN OPHIOPOGON EXTRACT FOR USE IN THE TREATMENT OF EYELIDS OR THE CONTOUR OF THE EYE

(71) Applicant: Laboratoires THEA, Clermont-Ferrand (FR)

(72) Inventors: Jacques PEYROT, Chamalieres (FR); Laurence ROBERTY, Pugey (FR); Michael ASSOULINE, Paris (FR)

(21) Appl. No.: 18/834,558

(22) PCT Filed: Feb. 13, 2023
(86) PCT No.: PCT/EP2023/053544

§ 371 Date: Jul. 30, 2024

(30) Foreign Application Priority Data
Feb. 14, 2022 (FR) FR2201284

(51) Int. Cl. A61K 36/8968; A61K 9/00; A61K 31/05; A61K 31/355; A61K 31/728; A61K 35/74; A61P 17/00

(52) U.S. Cl. CPC A61K 36/8968; A61K 9/0014; A61K 31/05; A61K 31/355; A61K 31/728; A61K 35/74; A61P 17/00

(57) ABSTRACT

A topical composition comprising, as active ingredient, a mixture of hyaluronic acid, trehalose and an extract of roots of Ophiopogon japonicus, for use as soothing and/or moisturizing agent for the eyelids and/or the contour of the eye in a subject with sensitive skin, in particular with blepharitis.

### TECHNICAL FIELD

The present invention relates to the technical field of ophthalmology.

It relates more particularly to a topical composition usable as soothing and/or moisturizing agent for the eyelids and/or the eye contour of a subject with sensitive skin, in particular skin with an atopic tendency, in particular the treatment of acute blepharitis or chronic blepharitis.

### PRIOR ART

The upper and lower eyelids as well as the eye contour have specific histological, biological and functional characteristics which make them extremely fragile and more sensitive than any other area of the face. For example, with more than 10,000 beats per day, the eyelids are extremely stressed. In addition, the eyelids, like the eye contour, are areas of the face that are not very rich in sebaceous glands; they have a thin and weakly protective hydrolipidic film, resulting in a reduced barrier function.

It is estimated that a third of the adult population has more vulnerable skin that reacts more strongly to its environment. This is the phenomenon of sensitive skin which is defined as skin hyperreactivity involving non-immunological mechanisms or a lowering of the skin's threshold of tolerance to stimuli that are usually well tolerated. The sensitivity of the skin is not the same on all areas of the body. For example, symptoms can appear only on the face. The manifestations of this sensitivity are sensations of discomfort, pulling sensations, sensations of warmth, tingling. Sensitive skin is often dry skin, which amplifies its sensitivity. The skin's hydrolipidic film (i. e., the skin's protective barrier) being defective, the skin is more vulnerable and reacts more strongly to its environment.

Blepharitis is one of the cutaneous manifestations of sensitive skin, in particular of skin with an atopic tendency, which is a chronic inflammatory condition of the skin with diverse origins. This skin sensitivity is also responsible for atopic dermatitis, contact dermatitis, contact eczema, psoriasis and even seborrheic dermatitis.

Blepharitis corresponds to inflammation of the eyelids. The causes of blepharitis are numerous. They can be due to dysfunction of the meibomian glands, oxidation of meibum lipids, oxidative stress of keratinocytes and cutaneous fibroblasts, or modification of the protective microbial biofilm. The causes can also be of environmental origin, in particular sensitivity of the eyelids to natural light, to blue light, as well as to pollution. They can be acute and chronic.
Acute blepharitis corresponds to sudden inflammatory outbreaks, appearing 24 to 48 hours after a triggering event: infection, airborne allergens, pollution, inadvertent direct or indirect contact with cosmetic products (eye makeup or nail polish for example).

Blepharitis becomes chronic when the patient has to deal with acute recurrences. The challenge is then to space out recurrences as much as possible and to reduce the sensations of discomfort and inflammation between two attacks.

Chronic blepharitis can also be the consequence of a specific ocular medical treatment, in particular a treatment against glaucoma, or a treatment against retinal diseases such as age-related macular degeneration, diabetic macular edema.

As mentioned in the preceding, blepharitis generates different phenomena such as inflammation, but also pruritus, secondary dryness, eyelid superinfection or bacterial proliferation.

The inflammation can manifest itself in different forms such as, for example, a thickened free edge, redness, a burning sensation, a sensation of heavy and swollen eyelids and itching in the evening.

Secondary dryness can cause discomfort, skin desquamation of the eyelids but also the appearance of crusts in the eyelashes.

Frequent chronicity of blepharitis is also observed, thus opening the way to 2 types of treatment, which are curative treatments with a view to discontinuation the signs of acute blepharitis and treatments acting on the chronicity of blepharitis, with the aim of spacing out recurrences.

The treatment of blepharitis is based on topical corticosteroids, which have the disadvantage of causing dependence.

The objective is therefore to offer effective care for the eyelids (lower and upper) and/or the eye contour, which is capable of.
calming sensations of discomfort and inflammation, reducing itching,
restoring the anti-evaporative functions of the Mebomian secretions,
effectively and lastingly soothing the eyelids and the eye contour,
lastingly moisturizing the eyelids and the eye contour,
healing the weakened eyelids and the eye contour,
restoring the barrier function of the skin of the eye and the eye contour,
rebalancing the microbiota by strengthening the natural defenses of the skin of the eye and the eye contour and limiting bacterial proliferation,
limiting the use of topical corticosteroids.

The document WO 2017/091916 describes a topical composition for the skin comprising a glycerol production promoter obtained from Staphylococcus epidermidis or keratinocytes. Trehalose, hyaluronic acid, and ADResyl (Ophiopogon japonicus root extract) are all three described as optional additives to the composition, each in a list comprising numerous compounds.

The document CN107693443 describes a lyophilized powder intended to be mixed with a solvent at the time of application to activate the ocular energy. The powder contains numerous constituents, including trehalose, hyaluronic acid, and ADResyl. The mixture is intended to be used in combination with yoga practice to reduce the effect of bags under the eyes, wrinkles, etc.

To the knowledge of the Applicant, no treatment fulfilling all of the aforementioned objectives for the treatment of the eyelids and the eye contour of a subject with sensitive skin, in particular skin with an atopic tendency, in particular the treatment of acute and chronic blepharitis, has been developed to date.

### DISCLOSURE OF THE INVENTION

One of the aims of the invention is therefore to propose a composition for use as a soothing and/or moisturizing agent, or in the soothing and/or moisturizing treatment for the eyelids and/or the eye contour of a subject with sensitive skin, in particular skin with an atopic tendency, in particular blepharitis, which makes it possible to stop the effects of this skin sensitivity, and in particular acute blepharitis, and which is capable of spacing out the acute recurrences characterizing blepharitis that has become chronic.

In other words, the aim of the invention is to develop a composition for the eyelids and/or the eye contour capable of causing a reduction, or even elimination, of the sensations linked to sensitive skin syndrome, that is to say itching, prickling, discomfort, tingling, irritation, scratching, sensations of heat, or even burning. In addition, the reduction in itching and scratching will limit, or even eliminate, the appearance of lesions and/or the dissemination of pathogens and therefore the contamination of the skin.

Another aim of the invention is to propose a composition for use as a soothing and/or moisturizing agent, or in the soothing and/or moisturizing treatment, for the eyelids and/or the eye contour of a subject with sensitive skin, in particular skin with atopic tendency, in particular blepharitis, which may be used, if appropiate, in addition to a corticosteroid treatment, so as to limit the doses of standard active ingredients used in these pathologies.

The Applicant demonstrated that the combination of a mixture of hyaluronic acid, trehalose and an extract of Ophiopogon japonicus roots was effective for its use as soothing and/or moisturizing agent, or in the soothing and/or moisturizing treatment, for the eyelids and/or the eye contour of a subject with sensitive skin, in particular skin with an atopic tendency, in particular acute blepharitis, and that the addition to this mixture of bakuchiol, a ferment extract of the microorganism Exiguobacterium sp. and a vitamin E derivative made it possible to act on its chronicity.

The Applicant demonstrated that the combination of a mixture of hyaluronic acid, trehalose and an extract of Ophiopogon japonicus roots was effective as soothing and/or moisturizing agent for the eyelids and/or the eye contour of a subject with sensitive skin, in particular skin with an atopic tendency, in particular acute blepharitis, and that the addition to this mixture of bakuchiol, a ferment extract of the microorganism Exiguobacterium sp., and a vitamin E derivative made it possible to act on its chronicicty.

More precisely, the Applicant demonstrated that taken separately, a mixture of hyaluronic acid and trehalose on the one hand and an extract of Ophiopogon japonicus roots on the other hand had an anti-inflammatory effect on the eyelids and/or or the contour of the eye and that, moreover and above all, the combination of these 3 elements had a superior effect on inflammation, justifying the existence of an unexpected synergy between the different elements.

In other words and according to a first feature, the invention relates to a topical composition comprising, as active ingredient, a mixture of hyaluronic acid, trehalose and an extract of Ophiopogon japonicus roots, for use as soothing and/or moisturizing agent for the eyelids and/or the eye contour in a subject with sensitive skin, in particular skin with atopic tendency, in particular blepharitis.

The Applicant noted that this combination made it possible to have an immediate effect on moisturization, by relieving and soothing the sensations of discomfort, pruritus, irritation, and by restoring the balance of the skin flora.

Within the meaning of the invention, a soothing agent ensures a reduction, or even elimination, of skin discomfort by reducing, or even eliminating, irritation, prickling, or itching.

Within the meaning of the invention, a moisturizing agent has in particular the effect of increasing the water content of the skin via several mechanisms such as the repair and/or lasting reinforcement of the skin barrier or the supply and/or the retention of water.

The effect is also lasting due to the restoration of the skin barrier, the reduction of the eyelid edema associated with inflammation, the strengthening of the natural defenses and the re-establishment of microbiotic homeostasis.

According to a first feature, the hyaluronic acid has an average molecular weight of between 100 kDa and 600 KDa, advantageously between 300 kDa and 450 KDa, preferably 300 kDa.

The selection of this specific molecular weight range makes it possible to have a hyaluronic acid that penetrates into the deep layers of the eyelid, thus making it possible to moisturize all the layers of the epidermis and also participating in the synthesis of natural hyaluronic acid by stimulating its production. This results in stimulation of the antibacterial activity of the skin due to the release of β defensins 2 by the skin.

In practice, hyaluronic acid represents between 0.01% and 1%, advantageously between 0.02% and 0.3% by weight of the composition.

In a preferred embodiment, and in particular when the composition is used with curative intent, the hyaluronic acid represents between 0.1% and 0.2%, preferably 0.15% by weight of the composition.

Trehalose appears to contribute to the overall effect of the composition through its bioprotective properties, thus preserving the cells of the epidermis. In practice, trehalose represents between 1% and 5%, preferably 3% by weight of the composition.

According to an essential feature, the composition comprises an extract of Ophiopogon japonicus roots.

The Applicant noted that particularly advantageous results were obtained in the treatment of blepharitis, in combination with other active ingredients, when the extract represented between 0.2% and 1% by weight of the composition.

In a preferred embodiment, and in particular when the composition is used with curative intent, the extract of Ophiopogon japonicus roots advantageously represents between 0.4% and 0.5% by weight of the composition, advantageously 0.45%.

According to another feature, when the composition is used to limit the chronicity of blepharitis, it further comprises bakuchiol, a ferment extract of the microorganism Exiguobacterium sp., and a water-soluble derivative of vitamin E.

The basic composition thus supplemented makes it possible to restore the barrier function of the skin and therefore to ensure long-term treatment of chronicized blepharitis. It also helps reduce sensations of scratching and irritation and protect the eyelids from external aggressions. It also reduces the appearance of dark circles and bags.

Under these conditions, it contains between 0.04% and 0.06%, advantageously 0.05% by weight of hyaluronic acid and between 0.5% and 0.7%, advantageously 0.6% by weight of Ophiopogon japonicus root extract.

Bakuchiol is extracted from babchi seeds and has functions similar to those of retinol. It thus helps regulate cell differentiation and stimulate collagen synthesis. It also acts as an antioxidant, maintains lipid homeostasis and inhibits opportunistic bacterial proliferation.

In practice, bakuchiol represents between 0.5% and 1%, preferably 0.75% by weight of the composition.

According to the invention, the composition contains a ferment extract of the microorganism Exiguobacterium sp., advantageously the raw material corresponding to the INCI: glycerin (and) water (and) hydrolyzed pea protein and glucose and sodium chloride and sodium succinate.

This raw material is known in particular to protect against attacks linked to light, aging (anti-blue shield) and hyperpigmentation (dark circle concealing effect). Here again, the Applicant had the idea of applying these properties to the particular case of the eyelid, by combining this product with the other aforementioned ingredients with which synergy is demonstrated.

In practice, the ferment extract of the microorganism Exiguobacterium sp. represents between 0.5% and 5%, preferably from 1.5% to 3% by weight of the composition.

For the treatment of recurrence, the composition also contains a water-soluble vitamin E derivative representing between 0.10% and T %, preferably 0.5% by weight of the composition.

The vitamin E derivative is known for its effect on protection against UVA/UVB irradiation, its protective effect against oxidative inflammation induced by UV and pollution, its effect on increasing ceramic synthesis and finally its dark circle concealing effect.

The composition of the invention can be used, if appropriate, in combination with topical corticosteroids, whether simultaneously or successively with them.

In an advantageous embodiment, the composition of the invention is used in addition to a treatment with topical corticosteroids, after discontinuation them, in particular for the treatment of acute blepharitis.

In another embodiment, the composition of the invention is used alternating with a treatment with topical corticosteroids, for example application of one in the evening and application of the other in the morning, in particular for the treatment of acute blepharitis.

In another embodiment, the composition of the invention is used alternating with a treatment with topical corticosteroids, for example application of one in the evening and application of the other in the morning, then use alone after discontinuation the topical corticosteroids, in particular for the treatment of acute blepharitis.

In another embodiment, the composition of the invention is used for the treatment of chronic blepharitis generated in the context of the treatment of an ocular disease, such as for example glaucoma, age-related macular degeneration, diabetic macular edema, in the absence of or in addition to topical corticosteroids.

### BRIEF DESCRIPTION OF THE FIGURES

Other advantages and characteristics of the invention will become clearer from the description which follows, given as non-limiting example, of the composition according to the invention, based on the appended drawings in which:

FIG. 1 is a curve representing the evolution of the percentage of HBD2 (β defensin 2) as a function of time after the application of different compositions to the eyelids following induction of inflammation by injection of LPS (lipopolysaccharide).

FIG. 2 is a curve representing the evolution of the daily self-assessment of itching, during the 28 days of application of the composition of the invention, by means of a score for itching ranging from 0 (no itching) to 10 (severe itching).

### DETAILED DESCRIPTION OF THE INVENTION

### Study of the Anti-Inflammatory Effect of the Composition of the Invention on the Eyelid in the Case of a Curative Treatment (Acute Blepharitis) by HBD2 Assay.

The list of ingredients used in the compositions tested is shown in Table 1.

TABLE 1

| | % of the total weight |
|---|---|
| Component | of the composition |
| Trehalose 100 (trehalose) | 3 |
| Primalhyal 300 (PM 300 hyaluronic acid) | 0.15 |
| ADResyl (Ophiopogon japonicus root extract) | 0.45 |

From these ingredients, three solutions were produced:
Solution 1: Primalhyal-Trehalose: mixture of 0.15% Primalhyal 300 and 3% Trehalose 100.
Solution 2: ADResyl: solution containing 0.45% ADResyl
Solution 3: (composition of the invention): solution 1+solution 2.
The solutions were prepared in distilled water.

The anti-inflammatory effect of the different solutions is observed by the ex vivo skin microdialysis technique on fresh eyelids. The principle of this technique is to insert into the dermis, a sterile microprobe provided at its end with a semi-permeable membrane continuously perfused with a sterile physiological saline. This membrane makes it possible, by concentration gradient, to analyze the dermal interstitial fluid, whether for endogenous compounds or for exogenous compounds.

3 eyelids are sampled and placed in an antibiotic bath in the form of a solution of 10% penicillin-streptomycin and 10% amphotericin B for 15 minutes. The samples are then dried using absorbent paper. A microdialysis probe is inserted into the dermis of each eyelid using a probe guide. The surviving eyelids are placed in nutrient medium on a hot plate. Waiting one hour allows the eyelids to stabilize following the insertion of the probe. A physiological solution (Ringer) is then perfused at 3 μL/min. The microdialysates are collected for 1 hour in order to obtain a baseline value (T0) of HBD-2. The induction of inflammation is obtained by the retroinjection of 0.8 mg/mL of LPS intradermally (12.5 μL on each side of the probe) and the compositions are applied to the surface of the eyelids. Samples are collected every hour for 6 hours. The microdialysates are stored at −20° C. until the HBD2 is assayed using an ELISA kit.

The HBD-2 contained in the samples binds specifically to the coated antibodies at the bottom of the wells. The revelation is carried out using a streptavidin-HRP solution and chromogen solutions which will cause development of a blue color. The color intensity is directly proportional to the amount of HBD-2 contained in the wells.

50 μL of standard (from 0 to 500 μg/mL of HBD-2) and 50 μL of microdialysate are placed in the wells of a 96-well plate with 100 μL of HRP-streptavidin. The plate is incubated for 1 hour at 37° C. After a series of washes, two different chromogen solutions are added to the wells and the plate is incubated for 15 min at 37° C. The color reaction is stopped by adding 50 μL of H₂SO₄. The plate is immediately read at 450 nm (Thermo Multiskan FC). The fluorescence intensity is proportional to the amount of HBD-2 present in the samples and standards. The set of calibration solutions allows determination of the HBD-2 concentrations in samples. The results are expressed as percentage of HBD-2 relative to T6 (6 h).

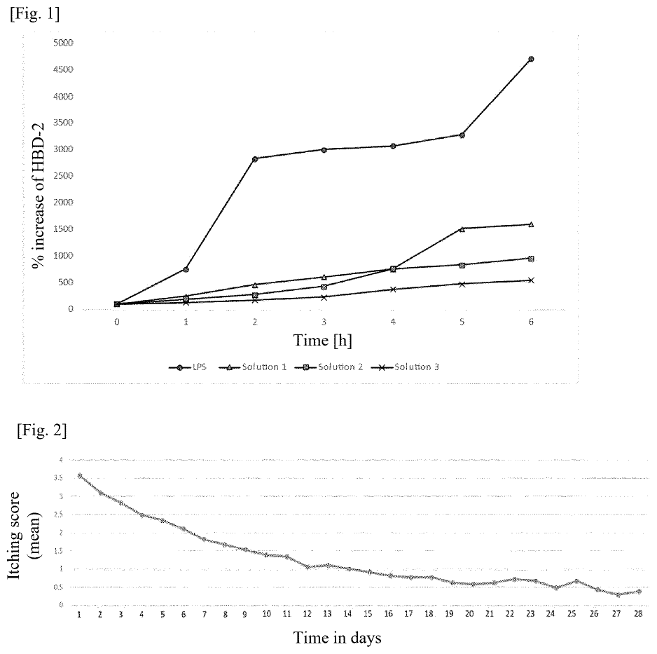

The results are shown in FIG. 1.

Solution 3 corresponding to the composition of the invention has an effective anti-inflammatory effect for the curative treatment of acute blepharitis induced in the present model by injection of LPS. Indeed, when a comparison is made with solution 1 (Primalhyal+Trehalose) and solution 2 (ADResyl alone), solution 3 very significantly reduces inflammation compared to solutions 1 and 2.

In Vivo Study of the Effect of the Composition of the Invention in Patients Suffering from Acute Blepharitis (Curative Treatment)

Composition 1 is prepared, the formula of which is shown in Table 2.

TABLE 2

| | Composition in |
|---|---|
| Name of the materials | % by weight |
| TREHALOSE 100 | 3.00 |
| AD-RESYL | 0.45 |
| PRIMALHYAL 300 | 0.15 |
| BIOXAN E 1300 IU (D-α-tocopherol) | 0.10 |
| OSMOSIS WATER | 83.50 |
| DERMOFEEL PA | 0.10 |
| LECIGEL | 2.00 |
| TEGOSOFT OER | 5.00 |
| OSMOSIS WATER | 5.00 |
| NaOH 10% aqueous | 0.70 |
| solution (Merck Chimie) | |
| TOTAL | 100.00 |

### Patient 1

### Profile:

24-year-old woman suffering from atopic blepharitis with edematous reaction of the eyelids.

### Protocol:

Treatment: topical corticosteroid in the evening and composition 1 applied to the eyelids in the morning
Discontinuation of the topical corticosteroid on the 3^{rd} day and continuation of the treatment with only composition 1 in the morning

### Observations:

Disappearance of the burning sensation on the 2^{nd} day
Disappearance of the edema from the 3^{rd} day
Control 5^{th} day: on examination, no desquamation

### Patient 2

### Profile:

18-year-old woman suffering from eye shadow contact dermatitis, edematous and pruritic blepharitis

### Protocol:

Treatment: topical corticosteroid in the evening and composition 1 applied to the eyelids in the morning

### Observations:

Sensation of soothing and comfort from the first application of composition 1.
Reduction by more than 90% of the edema on the 3^{rd} day
Control 5th day:
   Edema almost disappeared
   Mild furfuraceous desquamation

### Patient 3

### Profile:

50-year-old man: contact dermatitis with onctosis, unilateral edematous blepharitis developing more than 48 hours ago without signs of shingles

### Protocol:

Treatment: topical corticosteroid in the evening+composition 1 applied to the eyelids in the morning
Discontinuation of the corticosteroids after 48 hours+continuation of the treatment with composition 1 morning and evening

### Observations:

Almost immediate disappearance of burning sensations
Immediate and lasting sensation of comfort
Clear improvement in 48 hours
Reduction of the edema on the 3^{rd} day
Almost normal appearance of the affected eyelid on the 5^{th} day

### Patient 4

### Profile:

15-year old boy: blepharitis of both eyelids as a result of contact eczema.

### Protocol:

Right eye: Topical corticosteroid in the evening and Eczeane® UHT in the morning
Left eye: Topical corticosteroid in the evening and composition 1 in the morning

### Observations:

Right eye: continuation of topical corticosteroid on the 5^{th} day
Left eye: clear improvement on the 3^{rd} day with discontinuation of the topical corticosteroid and continuation of composition 1 alone.

### Conclusion

The above results show that the composition of the invention can replace topical corticosteroids or supplement them during treatment.

In Vivo Study of the Effect of the Composition of the Invention in Patients Suffering from Acute Blepharitis (Chronic Treatment)

Composition 2 is prepared, the formula of which is shown in Table 3.

TABLE 3

| | Composition in |
|---|---|
| Name of the materials | % by weight |
| TREHALOSE 100 | 3.00 |
| AD-RESYL | 0.60 |
| PRIMALHYAL 300 | 0.05 |
| SYTENOL A = bakuchiol | 0.75 |
| LUMICEASE ® BLUE = extract | 2.00 |
| of Exiguobacterium sp. | |
| TPNa = Vitamin E derivative | 0.50 |
| OSMOSIS WATER | 78.75 |
| NaOH 10% aqueous | 0.25 |
| solution (Merck Chimie) | |
| CARBOPOL ULTREZ 21 POLYMER | 0.10 |
| SIMULGEL NS | 4.00 |
| MASSOCARE MCT | 5.00 |
| LEXFEEL N20 | 5.00 |
| TOTAL | 100.00 |

### Patient 1

### Profile:

62-year old woman: on anti-glaucoma treatment (1 drop/day) with chronic blepharitis resulting from the treatment. The signs of chronic blepharitis are characterized by fine desquamation and accentuation of fine lines of the external cantus, sensation of skin discomfort with difficulty applying makeup.

### Protocol:

Morning and evening application of composition 2

### Observations:

### Control on the 10^{th} Day:

Sensation of comfort and soothing
The eyelids seem better moisturized according to the patient
Disappearance of redness
Ease of makeup application
On clinical examination, the eyelids are less red, less wrinkled, there is no more desquamation

### Control on the 30^{th} Day:

The patient confirms the subjective sensations observed on the 10th day with clear improvement of eyelid comfort
The tolerance is perfect
A lighter appearance of the eyelids is noted by the patient

### Control at the 3^{rd} Month:

The patient confirms the qualities of the product
She no longer suffers from blepharitis
Her fine lines are clearly reduced
The skin is significantly lighter

### Patient 2

### Profile:

72-year old woman: on anti-glaucoma treatment for 3 years (1 drop/day) with chronic blepharitis resulting from the treatment. The signs of chronic blepharitis are characterized by redness and desquamation, sensation of discomfort. Blepharitis is treated with a course of topical corticosteroid

### Protocol:

Morning and evening application of composition 2 and discontinuation of the topical corticosteroid

### Observations:

### Control on the 30^{th} Day:

The sensations of irritation have decreased
The patient feels comfort significantly greater than that she experienced before
On examination, the skin is less wrinkled, more toned and appears lighter, which is confirmed by the patient

### Patient 3

### Profile:

58-year old woman: on anti-glaucoma treatment for 5 years (1 drop/day) with erythematous and pigmentogenic blepharitis. Sensation of chronic discomfort with episodes of itching. Use of topical corticosteroids on demand despite contraindication.

### Protocol:

Discontinuation of topical corticosteroids
Morning and evening application of composition 2

### Observations:

### Control on the 30^{th} Day:

Rapid disappearance of sensations of itching, in 1 week
Restored eyelid comfort
Sensation of well-hydrated eyelids
Less visible pigmentation

### Patient 4

### Profile:

38-year old man: Atopic erythroderma, chronic blepharitis with lichenified appearance of the eyelids, disabling pruritus.

### Protocol:

Put on dupilumab which is a human monoclonal antibody targeting interleukins 4 and 13 and aimed at controlling atopic erythroderma after failure of other systemic treatments. Also put on emollient, face and body
Application of composition 2 morning and evening for 1 month on the eyelids.

### Observations:

### Control on the 30^{th} Day:

The patient says he had a sensation of soothing and suppleness of the eyelids on the 10 day
This sensation continues and increases on the 20^{th} and 30^{th} day
The eyelids are less stiff, much less scratchy
Blinking of the eyes is no longer painful
On examination, the eyelids are more supple, lighter, better moisturized
The effect is clearly superior to previous applications of conventional emollients
The patient who is very focused on his eyelids describes a very clear improvement
For 1 month he has no longer felt the need to use topical corticosteroids on his eyelids
Due to lack of product in the 6^{th} week, he is led to resume his conventional emollient

### Control at 10 Weeks:

Eyelid discomfort has reappeared
The general condition is improved but eyelid function is again degraded
The patient again uses a topical corticosteroid twice a week on the eyelids and the face.

### Conclusion

The observations show that the use of the composition of the invention gives effective and rapid results on chronic blepharitis, in particular with regard to redness, signs of desquamation, and the sensation of discomfort, in absence of or in addition to topical corticosteroids.

In Vivo Study of the Effect of the Composition of the Invention in Patients with Sensitive Skin
Composition 1 is prepared, the formula of which is shown in Table 2

Population/Profile: Men and women aged 18 and over all with skin sensitivity (i.e., sensitive skin) in the eyelids and the eye contour. This skin sensitivity is manifested as very dry skin which is then sensitive, irritated (pruritus) and causes discomfort in the subject.

### Protocol:

Morning and evening application of composition 1 (or 2) to both the eyelids and the eye contour.

### Observations:

Control 10 minutes (Dt10) after application and/or 28 days (Dj28) from the start of use of the composition of the invention of the scores of itching, irritation and discomfort making it possible to rule on the soothing, even moisturizing, effect of the composition of the invention.

### Results:

The results are shown in Table 4 and FIG. 2.

TABLE 4

| | | Variations | | | | |
|---|---|---|---|---|---|---|
| | | between the | Variations | | | |
| | | kinetics (mean ± | between | | | |
| | | standard | the data | Statistical | | |
| | Kinetics | deviation) | (%) | test | p-Value | Significance |
| Itching | ΔDt10 | −1.5 ± 0.3 | −23% | Student's t | <0.0001 | Yes (***) |
| (pruritus) | ΔDj28 | −6.5 ± 0.4 | −94% | Wilcoxon | <0.0001 | Yes (***) |
| score | | | | | | |
| Irritation | ΔDt10 | −1.0 ± 0.2 | −16% | Wilcoxon | <0.0001 | Yes (***) |
| Score | ΔDj28 | −6.1 ± 0.5 | −95% | Wilcoxon | <0.0001 | Yes (***) |
| Discomfort | ΔDt10 | −2.1 ± 0.5 | −30% | Wilcoxon | <0.0001 | Yes (***) |
| score | ΔDj28 | −6.4 ± 0.4 | −92% | Student's t | <0.0001 | Yes (***) |

### Conclusion

The results show that the use of the composition of the invention effectively soothes, in particular soothes and moisturizes, the skin of the eyelids and the eye contour of subjects with sensitive skin, in the absence of topical corticosteroids. This effect is observed in the very short term (+10 minutes) and in the medium/long term (+28 days), with a rapid and lasting reduction of itching sensations (FIG. 2) throughout the duration of use of the composition of the invention.

1-19. (canceled)

20. A topical composition for soothing the eyelids and/or eye contour of a subject having sensitive skin, the composition comprising:
hyaluronic acid;
trehalose; and
an Ophiopogon japonicus root extract.

21. The composition of claim 20, wherein the subject has skin with an atopic tendency.

22. The composition of claim 20, wherein the subject has blepharitis.

23. The composition of claim 22, wherein the blepharitis is acute blepharitis.

24. The composition of claim 23, wherein the composition is formulated to be co-administered with dermocorticoids, administered after stopping dermocorticoids, or administered alternately with dermocorticoids.

25. The composition of claim 22, wherein the blepharitis is chronic blepharitis.

26. The composition of claim 20, wherein the hyaluronic acid has an average molecular weight of between 100 kDa and 450 KDa.

27. The composition of claim 26, wherein the composition comprises between 0.01% and 1% hyaluronic acid by weight of the composition.

28. The composition of claim 27, wherein the composition comprises between 0.02 and 0.3% hyaluronic acid by weight of the composition.

29. The composition of claim 28, wherein the composition comprises between 0.1% and 0.2% hyaluronic acid by weight of the composition.

30. The composition of claim 20, wherein the composition comprises between 1% and 5% trehalose by weight of the composition.

31. The composition of claim 20, wherein the composition comprises between 0.2% and 1% Ophiopogon japonicus root extract by weight of the composition.

32. The composition of claim 31, wherein the composition comprises between 0.4% and 0.5% Ophiopogon japonicus extract by weight of the composition.

33. The composition of claim 20, wherein the composition further comprises bakuchiol and a water-soluble derivative of vitamin E.

34. The composition of claim 33, wherein the composition comprises between 0.04% and 0.06% by weight of hyaluronic acid, and between 0.5% and 0.7% by weight of Ophiopogon japonicus root extract.

35. The composition of claim 33, wherein the composition comprises between 0.5% and 1% bakuchiol by weight of the composition.

36. The composition of claim 33, wherein the bakuchiol is a ferment extract of the microorganism Exiguobacterium sp.

37. The composition of claim 36, wherein the ferment extract comprises glycerin, water, hydrolyzed pea protein, glucose, sodium chloride, and sodium succinate.

38. The composition of claim 37, wherein the composition comprises between 0.5% and 5% ferment extract.

39. The composition of claim 33, wherein the composition comprises between 0.1% and 1% water-soluble derivative of vitamin E by weight of the composition.

40. A method of treating an ocular disease, comprising administering the composition of claim 20 to a subject in need thereof.
